(19) United States
(12) Patent Application Publication
(10) Pub. No.: US 2018/0045642 A1
(43) Pub. Date: Feb. 15, 2018

(54) GAS DETECTION DEVICE AND METHOD FOR DETECTING GAS CONCENTRATION

(71) Applicant: RADIANT INNOVATION INC., Hsinchu City (TW)

(72) Inventors: TSENG-LUNG LIN, HSINCHU CITY (TW); SHAO-YUN YU, HSINCHU (TW); YU-CHIEN HUANG, HSINCHU CITY (TW)

(21) Appl. No.: 15/236,880

(22) Filed: Aug. 15, 2016

(51) Int. Cl. G01N 21/3504; G01N 33/00

(52) U.S. Cl. CPC G01N 21/3504; G01N 33/004

(57) ABSTRACT

The instant disclosure provides a gas detection device and method for detecting gas concentration. The gas detection device includes a chamber module, a light emitting module, an optical sensing module, and a light splitting module. The chamber module includes a light guiding chamber, a first sampling chamber, and a second sampling chamber. The light emitting module is disposed in the light guiding chamber to generate a projection light beam. The optical sensing module includes a first optical sensing unit disposed in the first sampling chamber, and a second optical sensing unit disposed in the second sampling chamber. The light splitting module is disposed in the chamber module. The projection light beam is split by the light splitting module to generate a first split light beam and a second split light beam.

### BACKGROUND

### 1. Technical Field

The instant disclosure relates to a gas detection device and method for detecting gas concentration, in particular, to a gas detection device and method for detecting gas concentration capable of measuring concentrations of different gases.

### 2. Description of Related Art

The carbon dioxide detection devices or carbon dioxide analyzing instruments in the market generally employ non-dispersive infrared (NDIR) absorption to detect the concentration of the gas. NDIR mainly utilizes calculation based on the Beer-Lambert law. The principle of such analysis is to detect the concentration of a specific gas by utilizing the absorption property of the gas toward infrared light having specific wavelength and the fact that the gas concentration is proportional to the absorption quantity. For example, carbon monoxide has a strongest absorption of a wavelength of 4.7 micron (μm) and carbon dioxide has a strongest absorption of a wavelength of 4.3 micron (μm).

However, the accuracy of the gas concentration detecting devices are limited to the structure of the gas sampling chamber and can only detect a specific concentration of the gas. Regarding the gas detection process employing NDIR, the absorption intensity of gas toward infrared is in positive correlation with the length and concentration. However, the gas sampling chamber of the existing gas concentration detecting devices is fixed and hence, when the length of the gas sampling chamber is too long and the concentration of the gas to be detected is too high, the gas having high concentration would absorb excessive infrared energy produced by the light emitting unit, and the light sensor unit cannot receive signals and is unable to detect the concentration of the gas. When the length of the gas sampling chamber is too short and the concentration of the gas to be detected is too low, the gas would absorb too little infrared energy, and the infrared energy generated by the light emitting unit would project onto the light sensor unit and would almost not be absorbed by the gas due to the short length of the gas sampling chamber. Moreover, when the infrared energy received by the light sensor unit is too low, the accuracy is reduced due to the noise.

Furthermore, the gas concentration detecting devices on the market can only detect one gas, i.e., they cannot detect a plurality of gases at the same time.

Therefore, there is a need for a device for detecting a plurality of gases or for detecting gases that have concentration with large differences, thereby overcoming the above disadvantages.

### SUMMARY

In view of the disadvantages of the existing art, the object of the instant disclosure is to provide a gas detection device and method for detecting gas concentration. The gas detection device and method for detecting gas concentration provided by the instant disclosure employ a single light emitting module to correspond to a plurality of light sensor units, thereby detecting a plurality of gases at the same time. The gas detection device and method for detecting gas concentration provided by the instant disclosure are also adapted to an environment having gases with different concentration having large differences.

An embodiment of the instant disclosure provides a gas detection device comprising a chamber module, a light emitting module, and optical sensing module and a light splitting module. The chamber module comprises a light guiding chamber, a first sampling chamber connected to the light guiding chamber and a second sampling chamber connected to the light guiding chamber. The light emitting module is disposed in the light guiding chamber, and the light emitting module is configured to generate a projection light beam. The optical sensing module comprises a first optical sensing unit disposed in the first sampling chamber, and a second optical sensing unit disposed in the second sampling chamber. The light splitting module is disposed in the chamber module. The projection light beam generated by the light emitting module is split by the light splitting module for forming a first split light beam projected onto the first optical sensing unit, and a second split light beam projected onto the second optical sensing unit.

Another embodiment of the instant disclosure provides a method for detecting gas concentration, comprising: providing a light emitting module, the light emitting module generates a first split light beam passing a first sampling chamber and projected onto a first optical sensing unit, the light emitting module generates a second split light beam passing a second sampling chamber and projected onto a second optical sensing unit, in which the size of the first sampling chamber is larger than the size of the second sampling chamber, the first sampling chamber has a first gas therein, and the second sampling chamber has a second gas therein; calculating a first tangent slope of a first curve equation based on a first split light beam energy received by the first optical sensing unit, and calculating a second tangent slope of a second curve equation based on a second split light beam energy received by the second optical sensing unit; and judging whether the absolute value of the first tangent slope is larger than the absolute value of the second tangent slope. When the absolute value of the first tangent slope is larger than or equal to the absolute value of the second tangent slope, outputting a concentration of the first gas. When absolute value of the first tangent slope is less than the absolute value of the second tangent slope, outputting a concentration of the second gas.

Yet another embodiment of the instant disclosure provides a method for detecting gas concentration, comprising: providing a light emitting module, the light emitting module generates a first split light beam passing a first sampling chamber and projected onto a first optical sensing unit, the light emitting module generates a second split light beam passing a second sampling chamber and projected onto a second optical sensing unit, wherein the size of the first sampling chamber is larger than the size of the second sampling chamber; calculating a concentration of a first gas in the first sampling chamber according to a first split light beam energy received by the first optical sensing unit, and calculating a concentration of a second gas in the second sampling chamber according to a second split light beam energy received by the first optical sensing unit; and judging whether the concentration of the first gas and the concentration of the second gas are larger than a predetermined threshold. When the concentration of the first gas and the concentration of the second gas are larger than a predetermined threshold, outputting the concentration of the second gas. When the concentration of the first gas and the concentration of the second gas are less than or equal to a predetermined threshold, outputting the concentration of the first gas.

The advantages of the instant disclosure reside in that by employing the light splitting module, the projection light beam generated by the light emitting module is split and forms a first split light beam projected onto the first optical sensing unit and a second split light beam projected onto the second optical sensing unit. The first optical sensing unit detects the property of a first gas and the second optical sensing unit detects the property of a second gas. In addition, the combination of the first optical sensing unit and the second optical sensing unit, and the first split light beam and the second split light beam generated by the projection light beam, the device and method of the instant disclosure can be adapted to environments in which the concentrations of different gases have large differences. In other words, the projection light beam generated by the light emitting module forms at least two split light beams for corresponding to at least two optical sensing units.

In order to further understand the techniques, means and effects of the instant disclosure, the following detailed descriptions and appended drawings are hereby referred to, such that, and through which, the purposes, features and aspects of the instant disclosure can be thoroughly and concretely appreciated; however, the appended drawings are merely provided for reference and illustration, without any intention to be used for limiting the instant disclosure.

### BRIEF DESCRIPTION OF THE DRAWINGS

The accompanying drawings are included to provide a further understanding of the instant disclosure, and are incorporated in and constitute a part of this specification. The drawings illustrate exemplary embodiments of the instant disclosure and, together with the description, serve to explain the principles of the instant disclosure.

FIG. 1 is one of the three-dimensional assembled views of the gas detection device of the first embodiment of the instant disclosure.

FIG. 2 is one of the three-dimensional exploded views of the gas detection device of the first embodiment of the instant disclosure.

FIG. 3 is a module block diagram of the gas detection device of the first embodiment of the instant disclosure.

FIG. 4 is a sectional schematic view taken along line IV-IV of FIG. 1.

FIG. 5 is one of the light beam projection schematic views of the gas detection device of the first embodiment of the instant disclosure.

FIG. 6 is another light beam projection schematic view of the gas detection device of the first embodiment of the instant disclosure.

FIG. 7 is sectional schematic view taken from line VII-VII in FIG. 1.

FIG. 8 is a sectional schematic view of another implementation of the gas detection device of the first embodiment of the instant disclosure.

FIG. 9 is a three-dimensional assembled schematic view of the gas detection device of the second embodiment of the instant disclosure.

FIG. 10 is the sectional schematic view taken along line X-X of FIG. 9.

FIG. 11 is one of the flow charts of the method for detecting gas concentration of the third embodiment of the instant disclosure.

FIG. 12 is one of the curve equation of the third embodiment of the instant disclosure.

FIG. 13 is another curve equation of the third embodiment of the instant disclosure.

FIG. 14 is another flow chart of the method for detecting gas concentration of the third embodiment of the instant disclosure.

FIG. 15 is a flow chart of the method for detecting gas concentration of the fourth embodiment of the instant disclosure.

### DESCRIPTION OF THE EXEMPLARY EMBODIMENTS

Reference will now be made in detail to the exemplary embodiments of the instant disclosure, examples of which are illustrated in the accompanying drawings. Wherever possible, the same reference numbers are used in the drawings and the description to refer to the same or like parts.

### First Embodiment

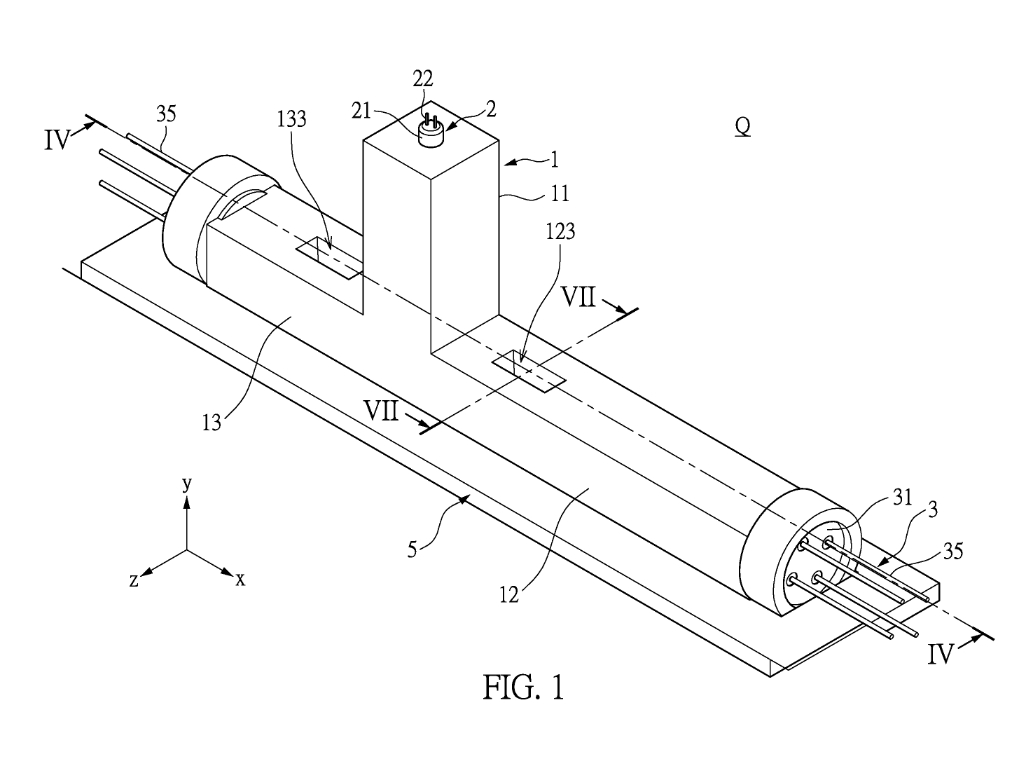

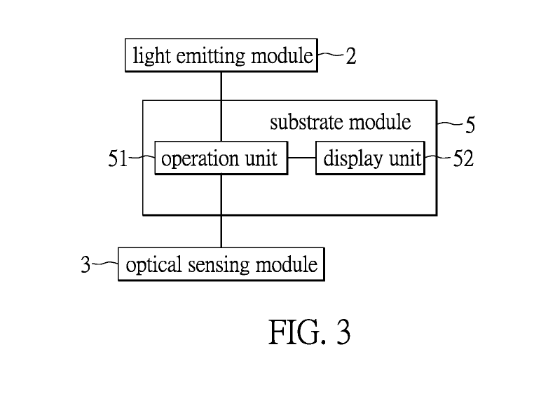

Please refer to FIG. 1 to FIG. 4. The first embodiment of the instant disclosure provides a gas detection device Q for detecting a concentration of a gas. The gas detection device Q comprises a chamber module 1, a light emitting module 2, an optical sensing module 3, a light splitting module 4, and a substrate module 5. The light emitting module 2 and the optical sensing module 3 are electrically connected on the substrate module 5. In addition, in FIG. 3, the substrate module 5 comprises a display unit 52 for displaying the concentration value of the gas and an operation unit 51 for calculating the concentration of the gas. The operation unit 51 is electrically connected to the display unit 52, the light emitting module 2 and the optical sensing module 3. In addition, for example, the light emitting module 2 can be an infrared light emitter for generating infrared light, the optical sensing module 3 is an infrared sensor such as a single-channel (single-beam) infrared sensor, or a double-channel infrared sensor (one of the infrared collecting windows is for detecting the gas concentration and another is for detecting the aging of the infrared light source, and both of which can calibrate each other). However, the instant disclosure is not limited thereto.

The gas detection device Q of the embodiments of the instant disclosure can detect the concentration or other properties of the gas to be measured. The gas to be measured can be carbon dioxide, carbon monoxide or the combination thereof. The instant disclosure is not limited thereto. In other words, by using a different light emitting module 2 and optical sensing module 3, it would be able to detect different types of gases. For example, the detection of the concentrations of different gases can be achieved by changing the wavelength filter on the optical sensing module 3.

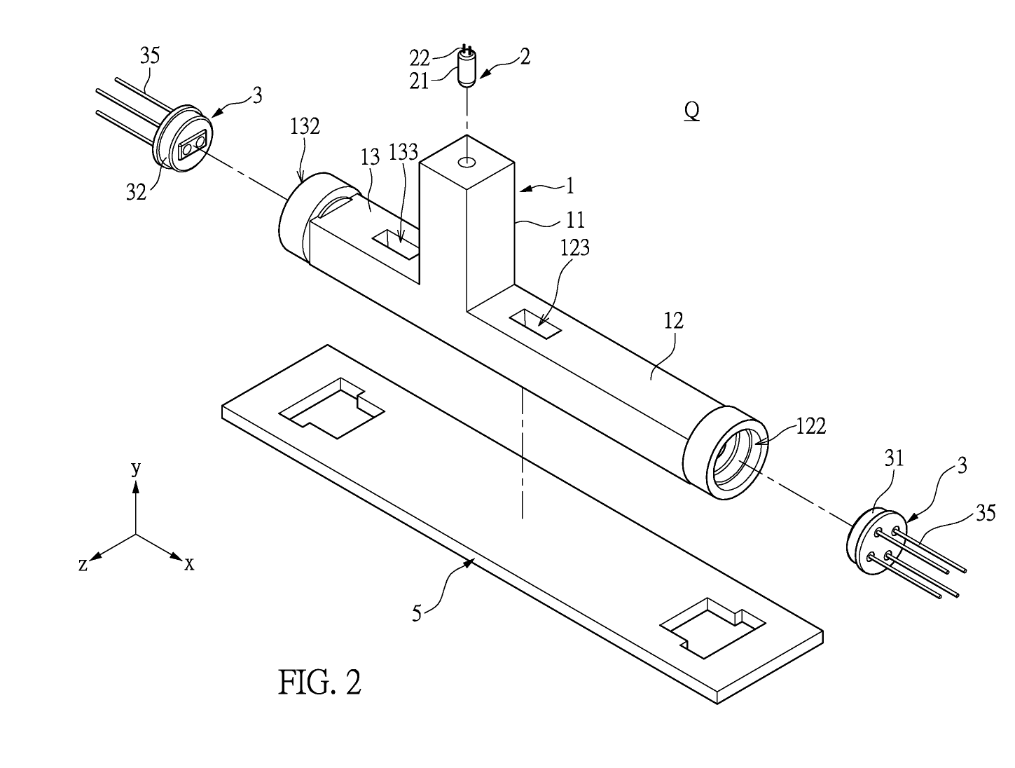

Next, please refer to FIG. 2 to FIG. 4. The chamber module 1 comprises a light guiding chamber 11, a first sampling chamber 12 connected to the light guiding chamber 11 and a second sampling chamber 13 connected to the light guiding chamber 11. The light guiding chamber 11 is disposed between the first sampling chamber 12 and the second sampling chamber 13. However, the instant disclosure is not limited thereto. In order to detect an environment in which the same gas has different concentrations with large differences, the size of the first sampling chamber 12 and the size of the second sampling chamber 13 are different. In the embodiments of the instant disclosure, the size of the first sampling chamber 12 is larger than the size of the second sampling chamber 13, i.e., the length of the first sampling chamber 12 is larger than the length of the second sampling chamber 13. However, the instant disclosure is not limited thereto. In other embodiments, the relationship between the sizes of the first sampling chamber 12 and the second sampling chamber 13 is not limited, as long as the first optical sensing unit 31 and the second optical sensing unit 32 can be used to detect a first gas and a second gas different from the first gas. In other words, the first optical sensing unit 31 is adapted to detect the properties of a first gas, and the second optical sensing unit 32 is adapted to detect the properties of a second gas different from the first gas. Therefore, an environment in which a same gas has very large different concentrations can be measured, or the properties of different gases can be detected, by employing a single light emitting module 2 corresponding to at least two optical sensing units.

For example, in the embodiments of the instant disclosure, the length direction of the first sampling chamber 12 (X direction) and the length direction of the light guiding chamber 11 (Y direction) are substantially perpendicular to each other. However, the instant disclosure is not limited thereto. In other words, in other embodiments, the length direction of the first sampling chamber 12 and the length direction of the second sampling chamber 13 can locate along the Z direction (for example, the length direction of the third sampling chamber 14 and the fourth sampling chamber 15 are both located along the Z direction as shown in the second embodiment). Moreover, in other embodiments, the length direction of the first sampling chamber 12 and the length direction of the second sampling chamber 13 are substantially parallel to the length direction of the light guiding chamber 11 (not shown), i.e., the length direction of the light guiding chamber 11, the length direction of the first sampling chamber 12 and the length direction of the second sampling chamber 13 are arranged along the Y direction.

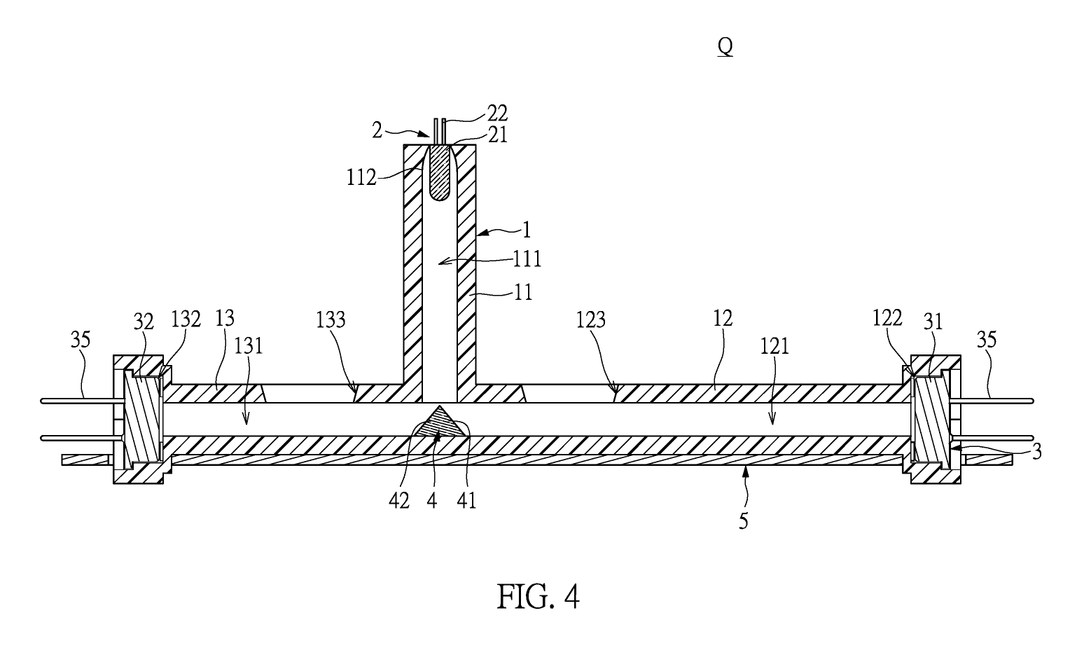

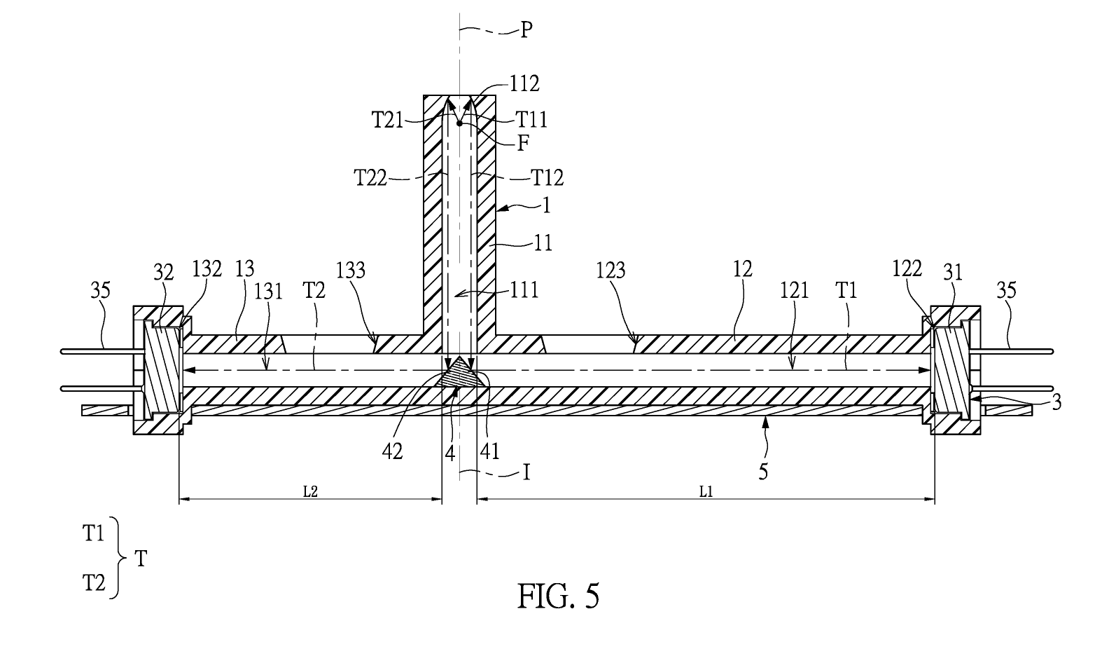

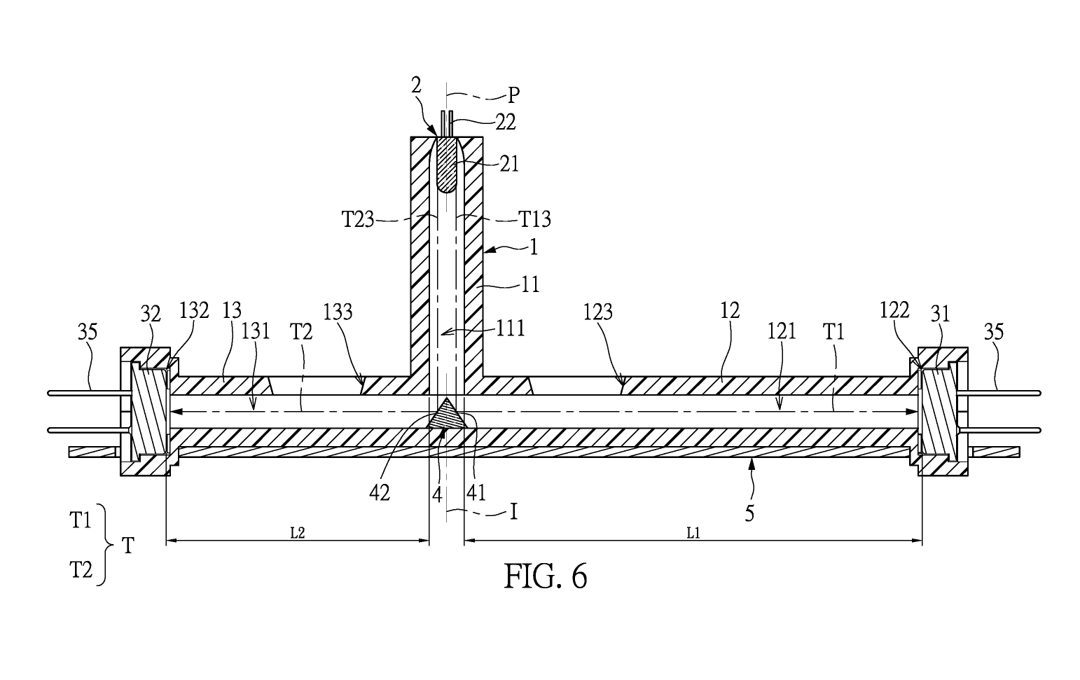

Next, as shown in FIG. 4, the light guiding chamber 11 has a light guiding space 111 and a reflective surface 112, the first sampling chamber 12 has a first sampling space 121 and a first receiving space 122, the second sampling chamber 13 has a second sampling space 131 and a second receiving space 132. The light guiding space 111, the first sampling space 121 and the second sampling space 131 are interconnected with each other. In addition, the light emitting module 2 is disposed in the light guiding chamber 11, the light emitting module 2 comprises a light emitting unit 21 and a connecting wire 22 electrically connected to the substrate module 5 (the connection between the connecting wire 22 and the substrate module 5 is not shown in the figure) for providing electrical energy to enable the light emitting unit 21 to generate a projection light beam T (please refer to FIG. 5 and FIG. 6) such as infrared light. In addition, the optical sensing module 3 comprises a first optical sensing unit 31 and a second optical sensing unit 32, the first optical sensing unit 31 is disposed in the first receiving space 122 and the second optical sensing unit 32 is disposed in the second receiving space 132 for receiving the projection light beam T generated by the light emitting unit 21. The connecting wire 35 of the optical sensing module 3 (the connecting wire 35 of the first optical sensing unit 31 and the connecting wire 35 of the second optical sensing unit 32) can be electrically connected with the substrate module 5 (the connection between the connecting wire 35 and the substrate module 5 is not shown in the figure). The instant disclosure does not limit how the light guiding space 111, the first sampling space 121 and the second sampling space 131 are intercommunicated with each other.

The first sampling space 121 of the first sampling chamber 12 and the second sampling space 131 of the second sampling chamber 13 are rectangular. However, the instant disclosure is not limited thereto. Each inner surface of the first sampling chamber 12 and the second sampling chamber 13 has a reflective layer (not shown) formed by metal plating or plastic plating. The reflective layer can be formed of gold-containing metal materials, nickel or the combination thereof. Therefore, the projection light beam T generated by the light emitting module 2 is repeatedly reflected in the first sampling space 121 and the second sampling space 131, thereby integrating the intensity of the projection light beam T generated by the light emitting module 2 and increasing the uniformity of the integrated light. The reflective surface of the light guiding chamber 11 can have a reflective layer for increasing the reflectance and increasing the amount of light projected onto the light splitting module 4.

Please refer to FIG. 4 to FIG. 6. The light splitting module 4 is disposed between the first sampling chamber 12 and the second sampling chamber 13, and the projection light beam T generated by the light emitting module 2 is split by the light splitting module 4 to form a first split light beam T1 projected onto the first optical sensing unit 31 and a second split light beam T2 projected onto the second optical sensing unit 32. For example, the light splitting module 4 comprises a first light splitting surface 41 and a second light splitting surface 42. Therefore, the projection light beam T generated by the light emitting unit 21 forms the first split light beam T1 projected onto the first optical sensing unit 31 and the second split light beam T2 projected onto the second optical sensing unit 32 by the first light splitting surface 41 and the second light splitting surface 42 respectively. The light splitting module 4 is not limited to the prism shown in the figures. In other embodiments, the light splitting module 4 utilizes a plurality of light splitters to form the first split light beam T1 and the second split light beam T2 from the projection light beam T generated by the light emitting unit 21.

As shown in FIG. 5, preferably, the reflective surface 112 of the light guiding chamber 11 is a paraboloid having a focus point F, and the light emitting unit 21 is disposed corresponding to the focus point F, i.e., the light emitting unit 21 is disposed on the focus point F and overlaps the focus point F. Therefore, a first projection light beam T11 and a second projection light beam T21 projected onto the light guiding chamber 11 can be uniformly reflected by the paraboloid and projected onto the light splitting module 4. In addition, in order to increase the reflectance of the paraboloid, a reflective layer described above can be disposed thereon.

Specifically, the projection light beam T comprises the first projection light beam T11 and the second projection light beam T21 projected onto the light guiding chamber 11, the first projection light beam T11 is reflected by the paraboloid of the light guiding chamber 11 and forms a first reflection light beam T12 projected onto the first light splitting surface 41 of the light splitting module 4, the first reflection light beam T12 is reflected by the first light splitting surface 41 and forms a first split light beam T1 projected onto the first optical sensing unit 31. The second projection light beam T21 is reflected by the light guiding chamber 11 and forms a second reflection light beam T22 projected onto the second light splitting surface 42 of the light splitting module 4, and the second reflection light beam T22 is reflected by the second light splitting surface 42 and forms a second split light beam T2 projected onto the second optical sensing unit 32.

In addition, as shown in FIG. 6, the projection light beam T generated by the light emitting unit 21 further comprises a first incident light beam T13 directly projected onto the first light splitting surface 41 of the light splitting module 4, and a second incident light beam T23 directly projected onto the second light splitting surface 42 of the light splitting module 4. The first incident light beam T13 is reflected by the first light splitting surface 41 and forms a first split light beam T1 projected onto the first optical sensing unit 31, and the second incident light beam T23 is reflected by the second light splitting surface 42 and forms a second split light beam T2 projected onto the second optical sensing unit 32.

In other words, the projection light beam T generated by the light emitting unit 21 comprises the first split light beam T1 projected onto the first optical sensing unit 31 and the second split light beam T2 projected onto the second optical sensing unit 32. The first split light beam T1 projected onto the first optical sensing unit 31 can be formed of the first projection light beam T11, the first reflection light beam T12 and the first incident light beam T13. The second split light beam T2 projected onto the second optical sensing unit 32 can be formed of the second projection light beam T21, the second reflection light beam T22 and the second incident light beam T23. When the light guiding chamber 11 is without the reflective surface 112, the first split light beam T1 projected onto the first optical sensing unit 31 can be directly formed by the first incident light beam T13, and the second split light beam T2 projected onto the second optical sensing unit 32 can be directly formed by the second incident light beam T23.

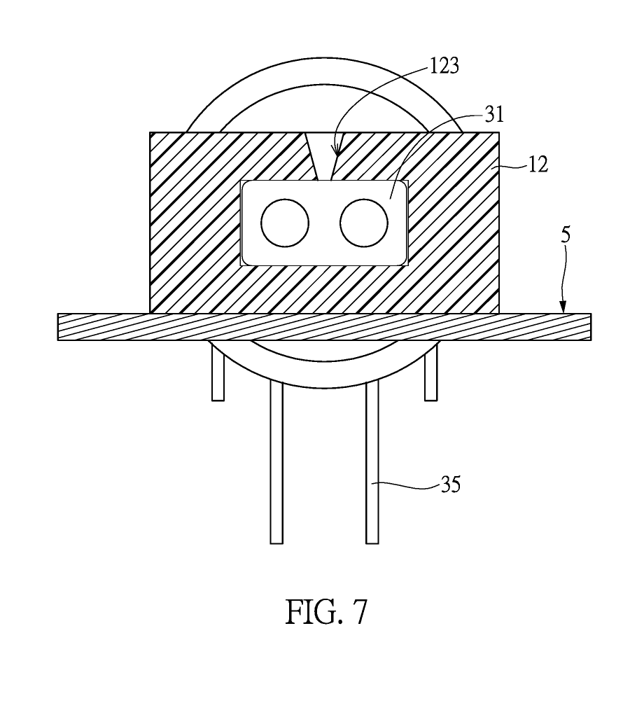

In addition, the first sampling chamber 12 further comprises a first gas diffusion tank 123 disposed thereon, and the second sampling chamber 13 further comprises a second gas diffusion tank 133 disposed thereon. The first gas diffusion tank 123 and the second gas diffusion tank 133 can be rectangular. The cross-section of the first gas diffusion tank 123 and the second gas diffusion tank 133 can be in a V-shape as shown in FIG. 5 to FIG. 7 and hence, the gas to be measured is subjected to Bernoulli's principle. Therefore, when the gas flows through the first gas diffusion tank 123 and the second gas diffusion tank 133 having a V-shape cross-section, the flow speed would increase since the diameter of the flow path changes, thereby increasing the diffusion of the gas and reducing the detecting time. A gas filtering membrane (not shown) can be further disposed on the first gas diffusion tank 123 and the second gas diffusion tank 133 to avoid the suspended particles in the gas to be measured from entering the first sampling space 121 and the second sampling space 131, causing internal pollution and affecting the detection accuracy.

In the embodiments of the instant disclosure, in order to detect environments in which the gases to be measured have concentrations with large differences, the first sampling chamber 12 has a first predetermined length L1, the second sampling chamber 13 has a second predetermined length L2, and the first predetermined length L1 of the first sampling chamber 12 is larger than the second predetermined length L2 of the second sampling chamber 13 Therefore, the first sampling chamber 12 is more suitable for detecting gases with low concentration, and the second sampling chamber 13 is more suitable for detecting gases with high concentration. In addition, since the first split light beam T1 and the second split light beam T2 received by the first optical sensing unit 31 and the second optical sensing unit 32 respectively are generated by the same light emitting unit 21, the detecting error is reduced.

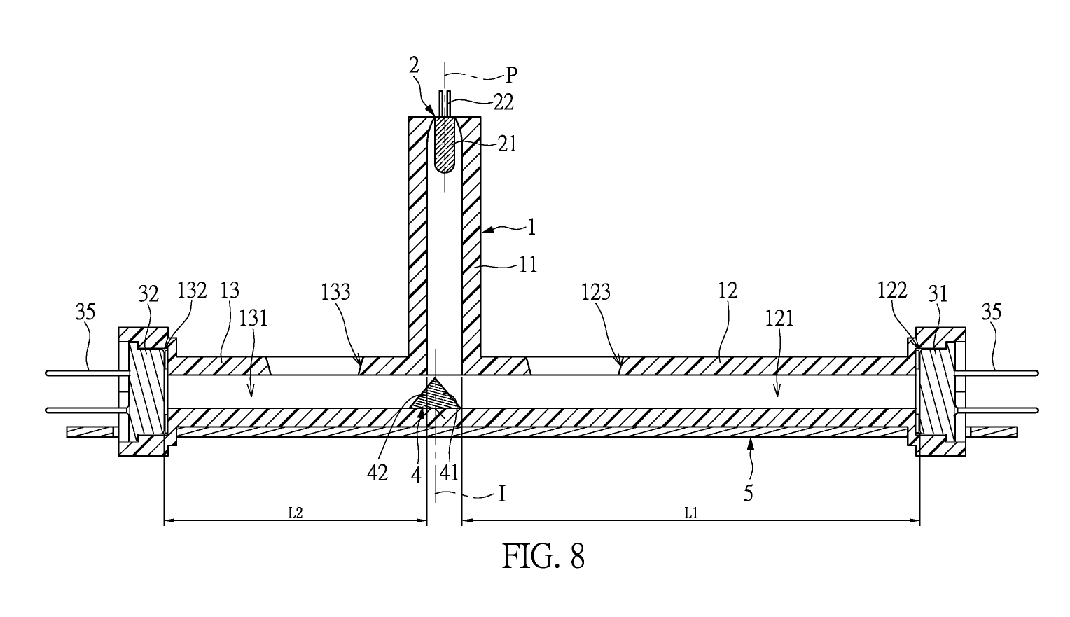

Next, please refer to FIG. 5, FIG. 6 and FIG. 8. By comparing FIG. 8 to FIG. 5, one can realize that in other embodiments, the location of the light splitting module 4 can be adjusted to adjust the light energy received by the first optical sensing unit 31 and the second optical sensing unit 32. Specifically, as shown in FIG. 5 and FIG. 6, the light guiding chamber 11 comprises a reflective surface 112 and a light axis P passing through a focus point F of the second light splitting surface 42, the light splitting module 4 has a center axis I between the first light splitting surface 41 and the second light splitting surface 42, and the center axis I passes through the light guiding space 111 and the light axis P overlaps with the center axis I. Alternatively, as shown in FIG. 8, the light axis P of the light guiding chamber 11 and the center axis I do not overlap with each other. In addition, in the present embodiment, since the projection light beam T and the first split light beam T1 are perpendicular to each other and the projection light beam T and the second split light beam T2 are perpendicular to each other, the first light splitting surface 41 and the center axis I has an included angle of 45 degrees, and the second light splitting surface 42 and the center axis I has an included angle of 45 degrees. However, the instant disclosure is not limited thereto.

### Second Embodiment

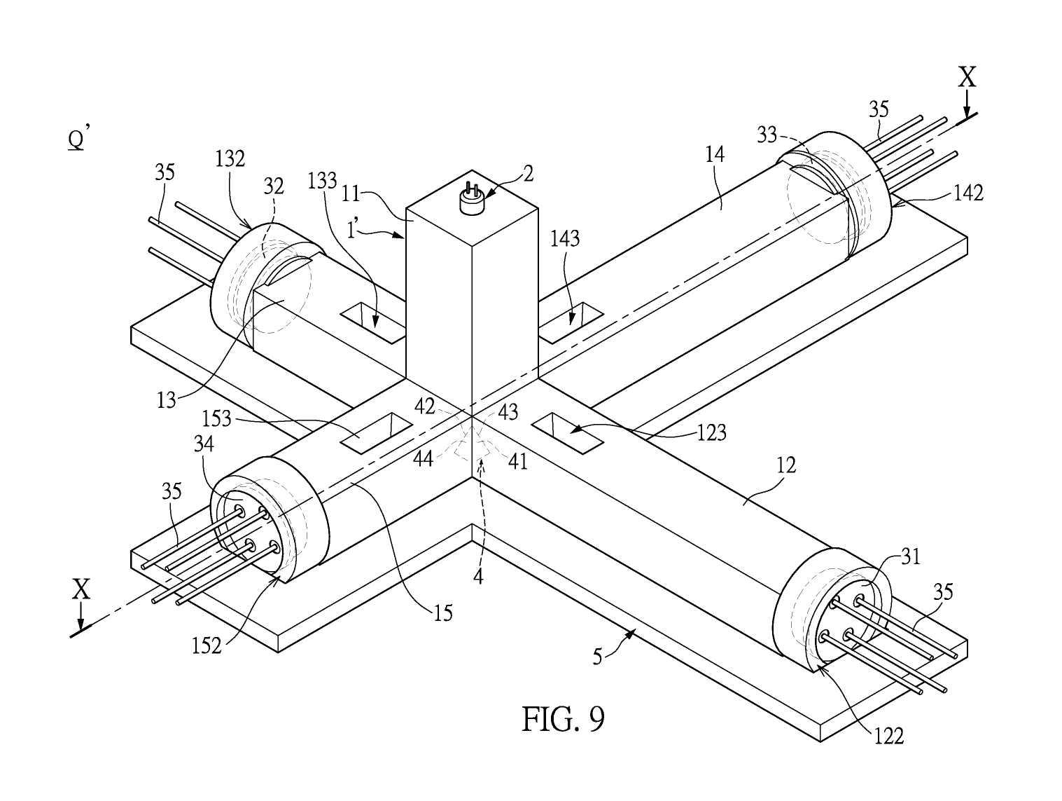

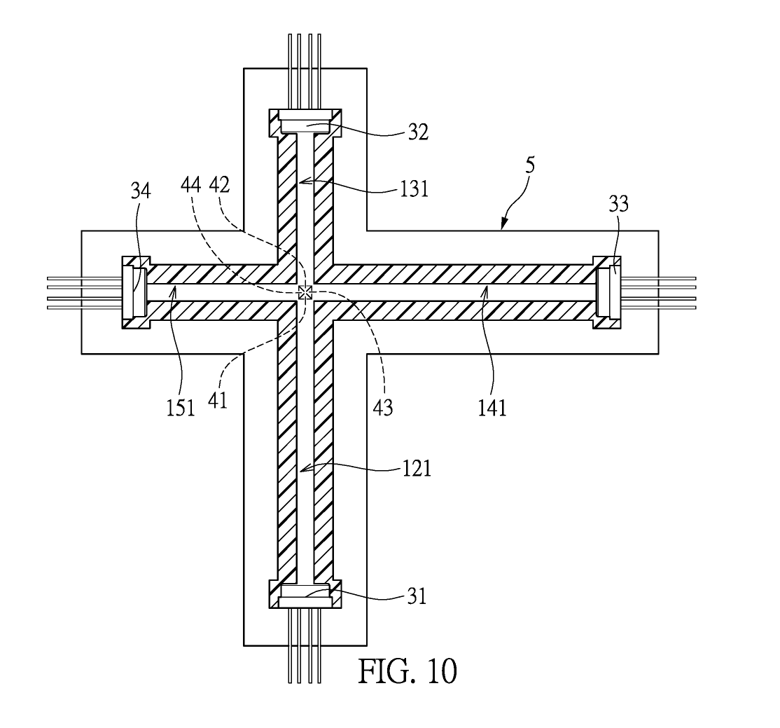

Please refer to FIG. 9 and FIG. 10. The second embodiment of the instant disclosure provides a gas detection device Q′. As shown in FIG. 9, the difference between the second embodiment and the first embodiment is that the chamber module 1′ provided by the second embodiment further comprises a third sampling chamber 14 connected to the light guiding chamber 11 and a fourth sampling chamber 15 connected to the light guiding chamber 11. The third sampling chamber 14 has a third sampling space 141 and a third receiving space 142, the fourth sampling chamber 15 has a fourth sampling space 151 and a fourth receiving space 152. The light guiding space 111, the third sampling space 141 and the fourth sampling space 151 are intercommunicated with each other. In other words, the third sampling space 141 and the fourth sampling space 151 are interconnected with the first sampling space 121 and the second sampling space 131. However, as long as the projection light beam T forms a plurality of split light beams (such as the first split light beam T1 and the first split light beam T1) projected onto a plurality of optical sensing units (such as the first optical sensing unit 31 and the second optical sensing unit 32), the sampling spaces are not limited to the structure described above. In other words, the sampling spaces can be interconnected with each other or do not interconnect with each other. In addition, the third sampling chamber 14 and the fourth sampling chamber 15 can further comprise a third gas diffusion tank 143 and a fourth gas diffusion tank 153 disposed thereon to facilitate the diffusion of the gas and reducing the detection time.

The light splitting module 4 further comprises a third light splitting surface 43 and a fourth light splitting surface 44, the optical sensing module 3 further comprises a third optical sensing unit 33 and a fourth sensing unit 34, the third optical sensing unit 33 is disposed in the third receiving space 142, the fourth sensing unit 34 is disposed in the fourth receiving space 152. Therefore, the projection light beam is split by the light splitting module 4 and forms a third split light beam projected onto the third optical sensing unit (not shown), and a fourth split light beam projected onto the fourth optical sensing unit.

The projection light beam comprises a third projection light beam and a fourth projection light beam (not shown) projected onto the light guiding chamber 11, the third projection light beam is reflected by the paraboloid of the light guiding chamber 11 and forms a third reflecting light beam (not shown) projected onto the third light splitting surface 43 of the light splitting module 4, the third reflecting light beam is reflected by the first light splitting surface 41 and forms a third split light beam projected onto the third optical sensing unit 33. In addition, the fourth projection light beam is reflected by the light guiding chamber 11 and forms a fourth reflecting light beam (not shown) projected onto the fourth light splitting surface 44 of the light splitting module 4, and the fourth reflecting light beam is reflected by the fourth light splitting surface 44 and forms a fourth split light beam projected onto the fourth sensing unit 34.

In addition, the projection light beam T further comprises a third incident light beam (not shown) directly projected onto the third light splitting surface 43 of the light splitting module 4, and a fourth incident light beam (not shown) directly projected onto the fourth light splitting surface 44 of the light splitting module 4. The third incident light beam is reflected by the third light splitting surface 43 and forms a third split light beam projected onto the third optical sensing unit 33, the fourth incident light beam is reflected by the fourth light splitting surface 44 and forms a fourth split light beam projected onto the fourth sensing unit 34.

The other structure features (such as the light guiding chamber 11, the first sampling chamber 12, the second sampling chamber 13, the light emitting module 2, the light splitting module 4 and the projection light beam T) of the second embodiment of the instant disclosure are similar to that of the previous embodiment and hence, are not described again herein. Therefore, by the addition of the third sampling chamber 14 and the fourth sampling chamber 15, the detecting range of the concentration of the gases can be increased, or the property of different gases can be detected (such as the concentrations of different gases).

### Third Embodiment

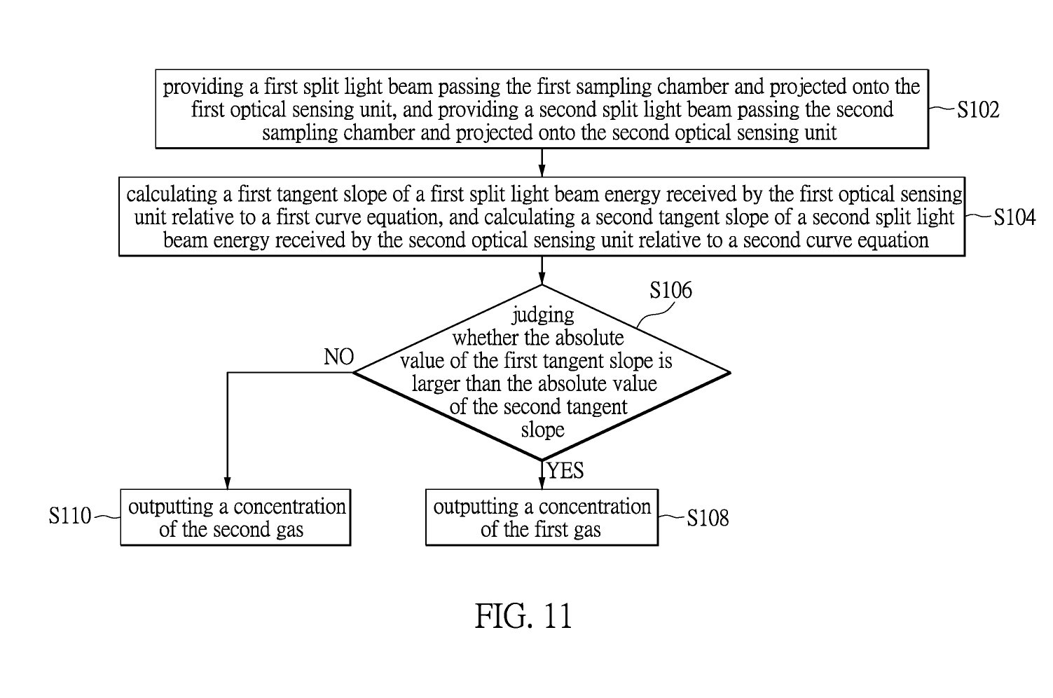

Please refer to FIG. 5, FIG. 6 and FIG. 11. The third embodiment of the instant disclosure provides a method for detecting gas concentration comprising the following steps. As shown in step S102: providing a first split light beam T1 passing the first sampling chamber 12 and projected onto the first optical sensing unit 31, and providing a second split light beam T2 passing the second sampling chamber 13 and projected onto the second optical sensing unit 32. Specifically, a projection light beam T can be generated by a light emitting module 2, and the projection light beam T passes through a light splitting module 4 and generates a first split light beam T1 and a second split light beam T2. In order to detect an environment in which the concentrations of the gases have large differences, the size of the first sampling chamber 12 is larger than the size of the second sampling chamber 13. For example, in the third embodiment, the first predetermined length L1 of the first sampling chamber 12 is four times of the second predetermined length L2 of the second sampling chamber 13, i.e., L1=4L2, in which L1 is the first predetermined length L1, L2 is the second predetermined length L2. In addition, in the third embodiment, the projection light beam T is an infrared beam, the first sampling chamber 12 has a first gas therein and the second sampling chamber 13 has a second gas therein. The first gas and the second gas in the third embodiment are the same type of gas (such as carbon dioxide, CO₂). However, the instant disclosure is not limited thereto.

Next, as shown in step S104: calculating a first tangent slope of a first split light beam energy received by the first optical sensing unit 31 relative to a first curve equation, and calculating a second tangent slope of a second split light beam energy received by the second optical sensing unit 32 relative to a second curve equation. Generally, in order to measure the concentration of the first gas and the second gas, the calculation can be carried out by the operation unit 51 in the substrate module 5 using the Beer-Lambert Law. Assuming I₀ is the energy of the infrared incident light (the initial energy of the infrared before being absorbed by the gas); Iₜ is the energy of the infrared received by the infrared light sensing unit (the energy received by the infrared light sensing unit after the infrared light being absorbed by the gas); K is the absorption coefficient; L is the length of the light path of the gas for absorbing light; C is the concentration of the gas. Based on the Beer-Lambert Law, the following equation is obtained:

Iₜ=I₀×exp×(−(L×K×C))

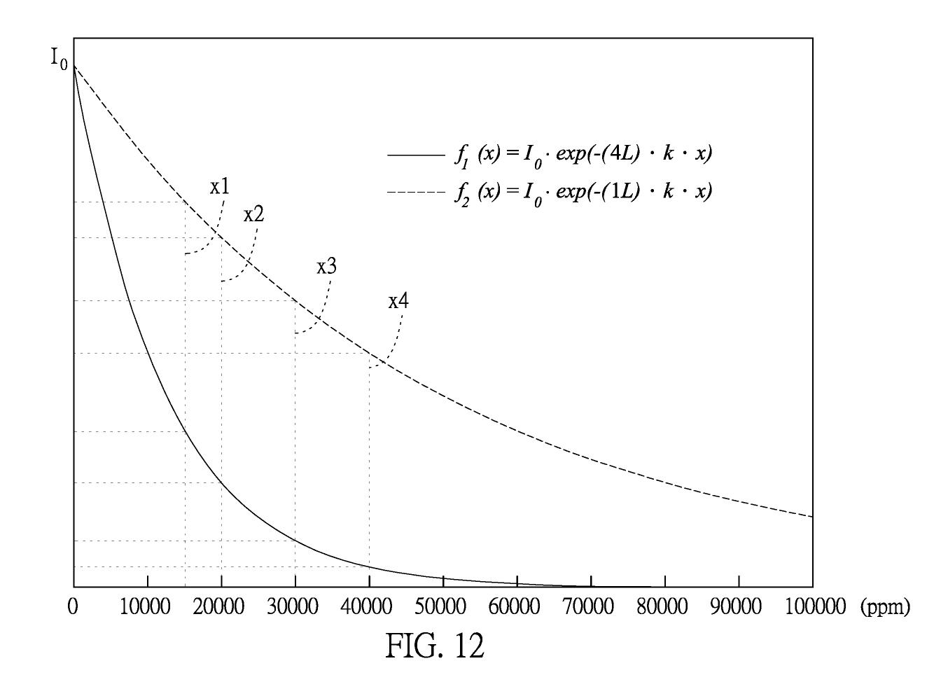

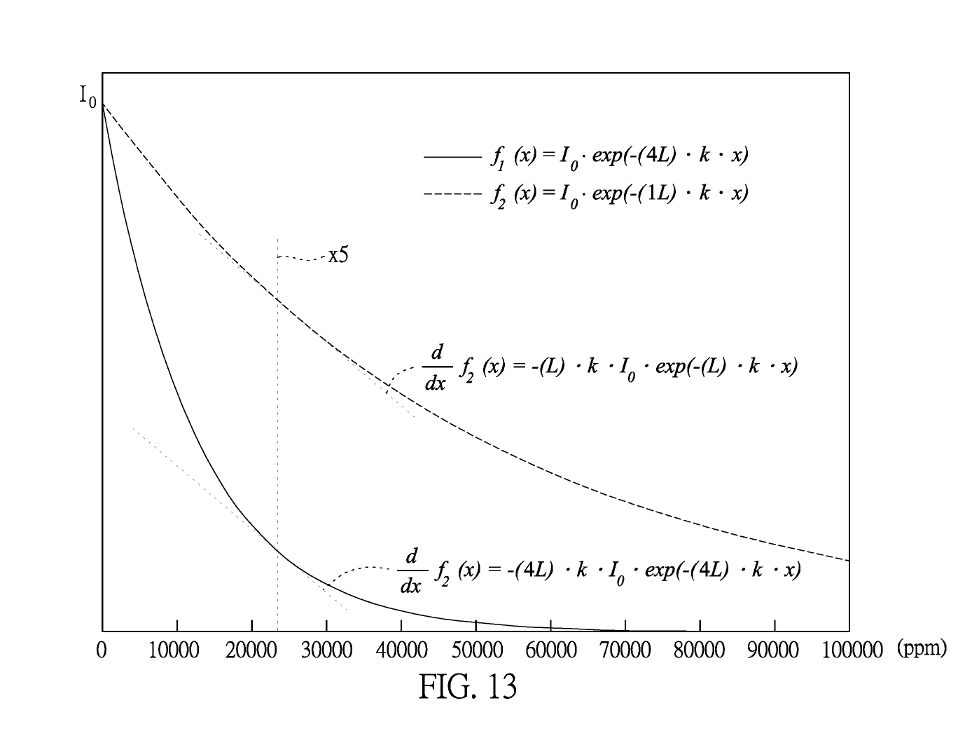

Next, please refer to FIG. 12 and FIG. 13. According to the Beer-Lambert Law, f₁(x) is defined as a first split light beam energy received by the first optical sensing unit 31 in the first sampling chamber 12, f₂(x) is defined as a second split light beam energy received by the second optical sensing unit 32 in the second sampling chamber 13. x is the concentration of the first gas or the second gas. In the present embodiment, the first predetermined length L1 of the first sampling chamber 12 is four times the second predetermined length L2 of the second sampling chamber 13 and hence, the concentration of the first gas in the first sampling chamber 12 and the concentration of the second gas in the second sampling chamber 13 can be calculated based on the following equation:

f₁(x)=I₀×exp×(−(4L×k×x)) (first curve equation)

f₂(x)=I₀×exp×(−(1L×k×x)) (second curve equation)

Specifically, the first curve equation and the second curve equation both satisfy the Beer-Lambert Law, and the operation unit 51 can calculate the concentration of a first gas in the first sampling chamber 12 based on a first split light beam energy received by the first optical sensing unit 31 and the first curve equation, and calculate the concentration of a second gas in the second sampling chamber 13 based on a second split light beam energy received by the second optical sensing unit 32 and the second curve equation. By obtaining the slope of the first curve equation and the second curve equation, one is able to judge whether the first optical sensing unit 31 or the second optical sensing unit 32 is able to obtain a larger infrared energy change in the same concentration interval.

As shown in FIG. 12, concentration intervals are used for description. The x1, x2, x3 and x4 in FIG. 12 represent different concentration values respectively. For example, the concentration value x1 is 15,000 ppm (parts per million), the concentration value of x2 is 20,000 ppm, the concentration value x3 is 30,000 ppm, and the concentration value x4 is 40,000 ppm. When the concentration of the first gas detected by the first optical sensing unit 31 and the concentration of the second gas detected by the second optical sensing unit 32 calculated by the operation unit 51 is between the concentration values x1 and x2, one is able to judge whether the first optical sensing unit 31 or the second optical sensing unit 32 can obtain a detecting value with more accuracy based on the calculation of a first tangent slope of the first curve equation between the concentration values x1 and x2, and a second tangent slope of the second curve equation between the concentration values x1 and x2.

Specifically, when the concentrations of the first gas and the second gas are between the concentration values x1 and x2, compared to the second curve equation, the first curve equation has more infrared energy change value for analyzing the concentration of the first gas having a concentration between the concentration values x1 and x2. In other words, the concentration value is more accurate when the infrared energy change is larger. Therefore, the first sampling chamber 12 is more suitable for the detection in the range of concentration value x1 to concentration value x2.

Alternatively, when the concentration of the first gas detected by the first optical sensing unit 31 and the concentration of the second gas detected by the second optical sensing unit 32 are between the concentration values x3 and x4, one is able to judge whether the first optical sensing unit 31 or the second optical sensing unit 32 can obtain a detecting value with higher accuracy based on the calculation of a first tangent slope of the first curve equation between the concentration values x3 and x4, and a second tangent slope of the second curve equation between the concentration values x3 and x4. Specifically, as shown in FIG. 12, when the concentration of the first gas and the concentration of the second gas are between the concentration values x3 and x4, compared to the first curve equation, the second curve equation has more infrared energy change value for analyzing the concentration of the first gas having a concentration between the concentration values x3 and x4. In other words, the concentration value is more accurate when the infrared energy change is larger. Therefore, the second sampling chamber 13 is more suitable for the detection in the range of concentration value x3 to concentration value x4.

As shown in FIG. 13, under a specific concentration value (x5), the first tangent slope of the first curve equation is equal to the second tangent slope of the second curve equation. In other words, the concentration value (x5) would be the judging factor for determining the use of the first sampling chamber 12 or the second sampling chamber 13. Therefore, the concentration value (x5) is a predetermined threshold. Under the concentration value x5, the first tangent slope is equal to the second tangent slope. The predetermined threshold x5 will be described in the following fourth embodiment. In addition, the first tangent slope of the first curve equation and the second tangent slope of the second tangent slope can be calculated by differentiation:

$$\frac{d}{dx} {f}_{1} ( x ) = - ( 4 L \times K ) \times {I}_{0} \times exp \times ( - ( 4 L \times k \times x ) )$$

$$\frac{d}{dx} {f}_{2} ( x ) = - ( L \times K ) \times {I}_{0} \times exp \times ( - ( L \times k \times x ) )$$

Please refer to FIG. 11. As shown in step S106: judging whether the absolute value of the first tangent slope is larger than the absolute value of the second tangent slope. Specifically, by judging the first tangent slope of the first curve equation and the second tangent slope of the second curve equation, one is able to judge which of the sampling chambers (the first sampling chamber 12 or the second sampling chamber 13) is suitable for detecting the concentration of the gas to be detected.

Next, as shown in step S108: outputting a concentration of the first gas. Specifically, when the absolute value of the first tangent slope is larger than the absolute value of the second tangent slope, the concentration of the first gas is smaller than the predetermined threshold x5, and the operation unit 51 can output the concentration of the first gas onto the display unit 52 for displaying the current concentration of the first gas. In other words, the current gas to be detected is suitable for being detected by the first sampling chamber 12. When the absolute value of the first tangent slope is equal to the absolute value of the second tangent slope, the concentration of the first gas can be output as well.

Next, as shown in step S110: outputting a concentration of the second gas. Specifically, when the absolute value of the first tangent slope is smaller than the absolute value of the second tangent slope, the concentration of the second gas is output. In other words, when the absolute value of the first tangent slope is smaller than the absolute value of the second tangent slope, the concentration of the second gas is larger than the predetermined threshold x5, and the operation unit 51 can output the concentration of the second gas onto the display unit 52 for displaying the current concentration of the second gas. In other words, the second sampling chamber 13 is suitable for detecting the current gas.

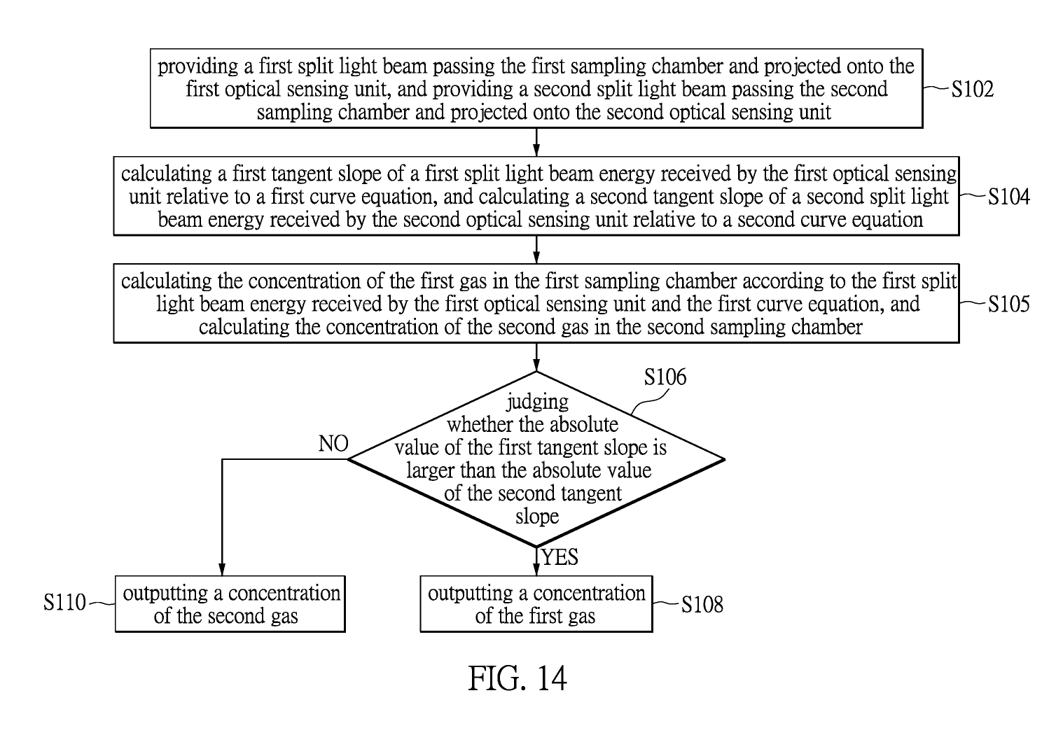

Next, please refer to FIG. 14. In another implementation, the method for detecting a gas concentration provided by the third embodiment of the instant disclosure further comprises step S105: calculating the concentration of the first gas in the first sampling chamber according to the first split light beam energy received by the first optical sensing unit and the first curve equation, and calculating the concentration of the second gas in the second sampling chamber. For example, the concentration of the first gas in the first sampling chamber 12 can be calculated according to the first split light beam energy received by the first optical sensing unit 31 and the first curve equation. Meanwhile, the concentration of the second gas in the second sampling chamber 13 can be calculated according to the second split light beam energy received by the second optical sensing unit 32 and the second curve equation. Therefore, the concentration of the first gas in the first sampling chamber 12 and the concentration of the second gas in the second sampling chamber 13 are optionally output onto the display unit 52.

Although step S105 is shown after step S104 in FIG. 14, the performing order of step S105 and step S104 is not limited in the instant disclosure. In other words, step S105 can be performed before the step of calculating the first tangent slope and the second tangent slope, during the step of calculating the first tangent slope and the second tangent slope or after the step of calculating the first tangent slope and the second tangent slope. In other words, step S105 and S104 can be performed independently. In addition, the first sampling chamber 12, the second sampling chamber 13, the light emitting module 2, the optical sensing module 3 and the substrate module 5 provided in the third embodiment are similar to that of the previous embodiments and are not described herein.

### Fourth Embodiment

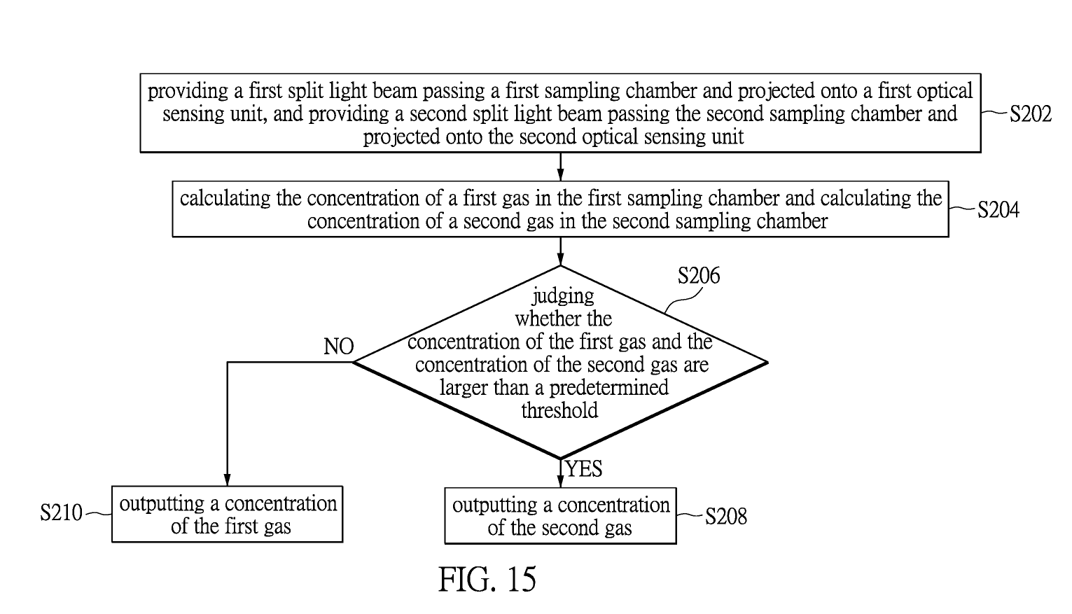

Please refer to FIG. 15. The fourth embodiment of the instant disclosure provides a method for detecting a gas concentration. As shown in FIG. 15, the main difference between the fourth embodiment and the third embodiment resides in that the method for detecting a gas concentration provided by the fourth embodiment involves directly judging whether the concentration of the first gas and the concentration of the second gas is larger than a predetermined threshold for determining which of the concentration of the first gas or the concentration of the second gas to be output.

Please refer to FIG. 13 and FIG. 15. The method for detecting the gas concentration provided by the fourth embodiment comprises the following steps. As shown in step S202, providing a first split light beam T1 passing a first sampling chamber 12 and projected onto a first optical sensing unit 31, and providing a second split light beam T2 passing the second sampling chamber 13 and projected onto the second optical sensing unit 32. Step S202 is similar to step S102 mentioned before and is not described in detail herein.

Next, as shown in step S204, calculating the concentration of a first gas in the first sampling chamber 12 and calculating the concentration of a second gas in the second sampling chamber 13. Specifically, the concentration of a first gas in the first sampling chamber 12 is calculated based on a first split light beam received by the first optical sensing unit 31, and the concentration of a second gas in the second sampling chamber 13 is calculated based on a second split light beam received by the second optical sensing unit 32. To be specific, as mentioned in the third embodiment, the concentration of the first gas is calculated based on the first split light beam energy and a first curve equation, and the concentration of the second gas is calculated based on the second split light beam energy and a second curve equation, and the first curve equation and the second curve equation satisfy the Beer-Lambert Law.

As shown in step S206, judging whether the concentration of the first gas and the concentration of the second gas are larger than a predetermined threshold x5. Specifically, the predetermined threshold x5 can be set according to the first tangent slope and the second tangent slope mentioned in the third embodiment. In other words, the predetermined threshold x5 satisfies the condition that the concentration of the first gas is equal to or close to (having an error that can be ignored) the concentration of the second gas, and that the first tangent slope of the concentration of the first gas relative to the first curve equation is equal or close to the second tangent slope of the concentration of the second gas relative to the second curve equation. For example, as shown in FIG. 13, at 23,000 ppm, the first tangent slope is equal to or close to the second tangent slope. Therefore, the predetermined threshold can be 23,000 ppm. However, the instant disclosure is not limited thereto. In other implementation, the first predetermined length L1 can be 3 centimeters (cm) to 6 centimeters for detecting carbon dioxide having a concentration value of 0˜50,000 ppm, and the second predetermined length L2 can be 2 centimeters to 3 centimeters for detecting carbon dioxide having a concentration value of more than 50,000 ppm. In other words, by adjusting the first predetermined length L1 of the first sampling chamber 12 and the second predetermined length L2 of the second sampling chamber 13, the predetermined threshold x5 can be changed. Therefore, one is able to detect environments with large gas concentration differences.

Next, as shown in step S208: outputting the concentration of the second gas. Specifically, when the concentration of the first gas and the concentration of the second gas are larger than the predetermined value x5, the concentration of the second gas is output. In other words, the absolute value of the first tangent slope is smaller than the absolute value of the second tangent slope, and the second sampling chamber 13 is suitable for detecting the current gas concentration. Therefore, operation unit 51 outputs the concentration of the second gas on the display unit 52 for displaying the concentration of the second gas.

Next, as shown in step S210: outputting the concentration of the first gas. Specifically, when the concentration of the first gas and the concentration of the second gas are smaller than or equal to the predetermined value x5, the concentration of the first gas is output. In other words, the absolute value of the first tangent slope is larger than the absolute value of the second tangent slope, and the first sampling chamber 12 is suitable for detecting the current gas concentration. Therefore, operation unit 51 outputs the concentration of the first gas on the display unit 52 for displaying the concentration of the first gas.

### Effectiveness of the Embodiments

In sum, the advantage of the instant disclosure is that by using the light splitting module 4, the gas detecting devices (Q, Q′) and the methods for detecting gas concentration provided by the embodiments, the instant disclosure is able to split the projection light beam T generated by the light emitting module 2 through the light splitting module 4 for forming a first split light beam T1 projected onto the first optical sensing unit 31 and a second split light beam T2 projected onto the second optical sensing unit 32. Therefore, the first optical sensing unit 31 can be used to detect the property of the first gas and the second optical sensing unit 32 can be used to detect the property of the second gas. In addition, based on the combination of the first optical sensing unit 31 and the second optical sensing unit 32, and the first split light beam T1 and second split light beam T2 generated by the projection light beam T, the gas detecting devices (Q, Q′) and the methods for detecting gas concentration provided by the embodiments of the instant disclosure are suitable for detecting environments having gases with large concentration differences.

Therefore, the projection light beam T generated by the light emitting module 2 forms at least two split light beams (the first split light beam T1, and the second split light beam T2) corresponding to at least two optical sensing units (the first optical sensing unit 31 and the second optical sensing unit 32). By using a plurality of split light beams (the first split light beam T1 and the second split light beam T2) formed by the same light emitting module 2, the accuracy of the concentration detection is increased and the cost is reduced. In addition, by setting the size of the first sampling chamber 12 larger than the size of the second sampling chamber 13, when the gas concentration is low, the first sampling chamber 12 with longer length can be used; when the gas concentration is high, the second sampling chamber 13 with shorter length can be used; and when the concentration is equal to or close to the predetermined threshold x5, the first sampling chamber 12 with longer length can be used (since the infrared energy received by the sensing unit is larger).

The above-mentioned descriptions represent merely the exemplary embodiment of the instant disclosure, without any intention to limit the scope of the instant disclosure thereto. Various equivalent changes, alterations or modifications based on the claims of the instant disclosure are all consequently viewed as being embraced by the scope of the instant disclosure.

What is claimed is:

1. A gas detection device comprising:
a chamber module comprising a light guiding chamber, a first sampling chamber connected to the light guiding chamber and a second sampling chamber connected to the light guiding chamber;
a light emitting module disposed in the light guiding chamber, the light emitting module is configured to generate a projection light beam;
an optical sensing module comprising a first optical sensing unit disposed in the first sampling chamber, and a second optical sensing unit disposed in the second sampling chamber; and
a light splitting module disposed in the chamber module;
wherein the projection light beam generated by the light emitting module is split by the light splitting module for forming a first split light beam projected onto the first optical sensing unit, and a second split light beam projected onto the second optical sensing unit.

2. The gas detection device according to claim 1, wherein the first sampling chamber and the second sampling chamber have different sizes.

3. The gas detection device according to claim 1, wherein the first optical sensing unit is configured to measure a property of a first gas, the second optical sensing unit is configured to measure a property of a second gas different from the first gas.

4. The gas detection device according to claim 1, wherein the light guiding chamber comprises a reflective surface, the reflective surface is a paraboloid having a focus point, and the light emitting unit corresponds to the focus point.

5. The gas detection device according to claim 1, wherein a length direction of the first sampling chamber and a length direction of the light guiding chamber are substantially perpendicular to each other, and a length direction of the second sampling chamber and the length direction of the light guiding chamber are substantially perpendicular to each other.

6. The gas detection device according to claim 1, wherein the light guiding chamber has a light guiding space, the first sampling chamber has a first sampling space and a first receiving space, the second sampling chamber has a second sampling space and a second receiving space, the first optical sensing unit is disposed in the first receiving space, the second optical sensing unit is disposed in the second receiving space, the light splitting module is disposed between the first sampling chamber and the second sampling chamber, the light splitting module comprises a first light splitting surface and a second light splitting surface.

7. The gas detection device according to claim 6, wherein the projection light beam comprises a first projection light beam and a second projection light beam projected on the light guiding chamber, the first projection light beam is reflected by the light guiding chamber for forming a first reflection light beam projected onto the first light splitting surface of the light splitting module, the first reflection light beam is reflected by the first light splitting surface for forming the first split light beam projected onto the first optical sensing unit, the second projection light beam is reflected by the light guiding chamber for forming a second reflection light beam projected onto the second light splitting surface of the light splitting module, the second reflection light beam is reflected by the second light splitting surface for forming the second split light beam projected onto the second light sensing unit.

8. The gas detection device according to claim 6, wherein the projection light beam comprises a first incident light beam projected onto the first light splitting surface of the light splitting module and a second incident light beam projected onto the second light splitting surface of the light splitting module, the first incident light beam is reflected by the first light splitting surface for forming the first split light beam projected onto the first optical sensing unit, the second incident light beam is reflected by the second light splitting surface for forming the second split light beam projected onto the second optical sensing unit.

9. The gas detection device according to claim 6, the chamber module further comprises a third sampling chamber connected to the light guiding chamber and a fourth sampling chamber connected to the light guiding chamber, the third sampling chamber has a third sampling space and a third receiving space, the fourth sampling chamber has a fourth sampling space and a fourth receiving space, the light splitting module further comprises a third light splitting surface and a fourth light splitting surface, the optical sensing module further comprises a third optical sensing unit and a fourth optical sensing unit, the third optical sensing unit is disposed in the third receiving space, the fourth optical sensing unit is disposed in the fourth receiving space.

10. The gas detection device according to claim 1, wherein the light guiding chamber comprises a reflection surface and a light axis passing a focus point of the reflection surface, the light splitting module has a center axis located between the first light splitting surface and the second light splitting surface, the center axis passes through the light guiding space and coincides with the center axis or does not coincide with the center axis.

11. A method for detecting gas concentration, comprising:
providing a light emitting module, the light emitting module generates a first split light beam passing a first sampling chamber and projected onto a first optical sensing unit, the light emitting module generates a second split light beam passing a second sampling chamber and projected onto a second optical sensing unit, wherein the size of the first sampling chamber is larger than the size of the second sampling chamber, the first sampling chamber has a first gas therein, and the second sampling chamber has a second gas therein;
calculating a first tangent slope of a first curve equation based on a first split light beam energy received by the first optical sensing unit, and calculating a second tangent slope of a second curve based on a second split light beam energy received by the second optical sensing unit; and
judging whether the absolute value of the first tangent slope is larger than the absolute value of the second tangent slope;
wherein when the absolute value of the first tangent slope is larger than or equal to the absolute value of the second tangent slope, outputting a concentration of the first gas;
wherein when absolute value of the first tangent slope is less than the absolute value of the second tangent slope, outputting a concentration of the second gas.

12. The method according to claim 11, further comprising:
calculating the concentration of the first gas in the first sampling chamber according to the first split light beam energy received by the first optical sensing unit and the first curve equation, and calculating the concentration of the second gas in the first sampling chamber according to the second split light beam energy received by the second optical sensing unit and the second curve equation.

13. The method according to claim 11, wherein a projection light beam generated by the light emitting module is split by a light splitting module for forming the first split light beam and the second split light beam, and the first curve equation and the second curve equation satisfy the Beer-Lambert law.

14. A method for detecting gas concentration, comprising:
providing a light emitting module, the light emitting module generates a first split light beam and a second light beam, the first split light beam passes a first sampling chamber and is projected onto a first optical sensing unit, and the second split light beam passes a second sampling chamber and is projected onto a second optical sensing unit, wherein the size of the first sampling chamber is larger than the size of the second sampling chamber;
calculating a concentration of a first gas in the first sampling chamber according to a first split light beam energy received by the first optical sensing unit, and calculating a concentration of a second gas in the second sampling chamber according to a second split light beam energy received by the second optical sensing unit; and
judging whether the concentration of the first gas and the concentration of the second gas are larger than a predetermined threshold;
wherein when the concentration of the first gas and the concentration of the second gas are larger than the predetermined threshold, outputting the concentration of the second gas;
wherein when the concentration of the first gas and the concentration of the second gas are less than or equal to the predetermined threshold, outputting the concentration of the first gas.

15. The method according to claim 14, wherein the concentration of the first gas is calculated by the first split light beam energy and a first curve equation, the concentration of the second gas is calculated by the second split light beam energy and a second curve equation, the first curve equation and the second curve equation satisfy the Beer-Lambert law, the predetermined threshold is a concentration satisfied by a condition that the concentration of the first gas is equal to or substantially equal to the concentration of the second gas and that a first tangent slope of the concentration of the first gas relative to the first curve equation is equal to or substantially equal to a second tangent slope of the concentration of the second gas relative to the second curve equation.
